(19) United States
(12) Patent Application Publication
(10) Pub. No.: US 2006/0183703 A1
(43) Pub. Date: Aug. 17, 2006

(54) Circular expression construct for gene therapeutic applications

(76) Inventors: Matthias Schroff, Berlin (DE); Colin Smith, Corvalis, OR (US)

Correspondence Address: NILS H. LJUNGMAN;NILS H. LJUNGMAN & ASSOCIATES, P.O. BOX 130, GREENSBURG, PA, 15601-0130, US

(21) Appl. No.: 11/299,327

(22) Filed: Dec. 9, 2005

Related U.S. Application Data
(63) Continuation-in-part of application No. PCT/DE03/01970, filed on Jun. 10, 2003.

(51) Int. Cl. A61K 48/00; C12P 19/34

(52) U.S. Cl. 514/44; 435/91.2

(57) ABSTRACT

Method for producing a circular minimalist expression construct closed in an annular manner, from a double-strand DNA, an expression construct produced according to said method, and the use of the same in gene therapy and vaccination. The abstract of the disclosure is submitted herewith as required by 37 C.F.R. §1.72(b). As stated in 37 C.F.R. §1.72(b): A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims. Therefore, any statements made relating to the abstract are not intended to limit the claims in any manner and should not be interpreted as limiting the claims in any manner.

### CONTINUING APPLICATION DATA

This application is a Continuation-In-Part application of International Patent Application No. PCT/DE2003/001970, filed Jun. 10, 2003. International Patent Application No. PCT/DE2003/001970 was pending as of the filing date of this application. The United States was an elected state in International Patent Application No. PCT/DE2003/001970.

### BACKGROUND

1. Technical Field

This application relates to a method for producing a minimal expression construct out of a circular, annular closed, DNA double-strand, as well as the produced expression construct itself. Such expression constructs (vectors) should be used especially in the field of gene therapy. Gene therapy means the introduction of one or more ectopic genes into the organism to produce a therapeutic effect for the organism.

2. Background Information

Gene therapy depends on the development of relatively harmless and easy to use in-vivo gene transfer methods, whether for allowing an efficient and stable gene expression in definite organs or for an intended inhibition of protein expression of specific genes.

Because of the numerous barriers (plasma membranes, endosomes and cell core) during transfer of genetic material into the cell, the transfection of DNA is a rare and relatively unpredictable process. Thus, the insufficient transfection efficiency is a major problem of so far developed vectors, because by injection of DNA into tissues the bigger part of cells will not be transfected. Moreover, successfully transfected cells express the transfected DNA sequences differently.

Basically it has to be distinguished between viral and non-viral gene transfer methods. Viral transfer methods use genetically modified viruses as transport vehicles. Because wild type viruses and their derived vectors also have, besides their high transfection efficiency, good tissue specificity, they are generally mostly used these days for gene transfer.

But the use of such viruses in gene therapy poses security risks that cannot be underestimated, which risks are massively opposed to their use in gene therapy. For example, the possibility of recombination of the introduced viral particles with naturally present viruses in a patient represents an inherent security risk. From this uncontrollable recombination of reproducible and not-reproducible viruses, new and pathogenic hybrid viruses can arise. Moreover, immunogenic reactions, caused by anti-vector immunity, are a serious side effect that can accompany the use of viral vectors. For example, the application of high doses of an adenovirus led in a clinical trial to the death of the patient; the obvious reason for this was a strong overreaction of the immune system (Lehrman, 1999, Nature 401: 517-518). The cases of leukemia diseases after gene therapy demonstrate that such problems are not solved these days and represent a serious step backwards in gene therapy (Buckley R H, 2002, Lancet 360: 1185-6).

These disadvantages are essentially avoided by the use of non-viral gene transfer systems usually derived from plasmids and which are also designated as “naked” DNA. But they have, besides much lower transfection rates and accompanying lower expression rates of the transferred sequences, also a missing cell or tissue specificity.

A further disadvantage of known, non-viral gene transfer systems is the relatively great portion of bacterial DNA sequences that are contained in these plasmids. These bacterial DNA sequences can cause serious problems in the target organism. So naturally contained immune stimulatory sequences (“ISS”, e.g. unmethylated cytosine-guanine dinucleotides, “CpG”) of plasmids lead to a stimulation of effector cells of the immune systems, and, consequently, to the distribution of inflammatory cytokines and interferons (Krieg, 2002, Annu Rev Immunol 20:709-60). Therefore, in all cases where the induction of an immunologic Th1-phenotype is unwanted or even contra-indicated, this should be avoided. To these cases belong all diseases with autoimmune components, as the systemic Lupus Erythematosus or Morbus Crohn as well as numerous indications for gene therapy, working without participation of the immune system, as the metabolism disease Mucoviscidosis or the alpha-1-antitrypsin deficiency.

Furthermore, the known plasmids contain antibiotic resistance genes, which are necessary for their selection. The consequence of the possibility of recombination with ubiquitary present bacteria of the organism is the danger of an increase of antibiotic resistant bacteria. This spreading of antibiotic resistance, with regard to the several times the application of the therapeutic genes is necessary, is a serious problem and, for this reason, is not justifiable.

Coutelle et al. were able to produce by reduction of the bacterial DNA sequences within plasmids so-called mini-circle DNA, which led in in vitro experiments to up to tenfold higher expression rates in comparison to conventional plasmids (Coutelle et al., 2001, J of Biol. Chem. 25: 23018-23027). Plasmids were used containing two recognition sequences for recombinase. Because of the bacterial origin of these recombinase sites, these vectors contain rests of bacterial DNA, so that the disadvantages of common plasmids are reduced, but still present. A targeted gene transfer with these mini-circles is also not possible since a specific and controllable binding of transfer mediating ligands is impossible because of the structure of the mini-circle.

The U.S. Pat. No. 6,143,530 also describes plasmids with a minimized portion of DNA with bacterial origin. But the production process also needs bacterial recombinase and this is the reason for the presence of bacterial DNA sequences, which are capable of causing vector-induced, inflammatory processes.

In the U.S. Pat. No. 6,265,218, a method for producing a vector without a selection marker gene is described. The vectors produced according to the described method are designed for gene therapy or the production of pharmaceutical medicines for gene therapy. The only feasible vectors described in this document are produced using a recombinase system. This inevitably causes expression constructs containing vector sequences.

Another kind of non-viral vectors are minimal, partly double stranded closed expression constructs. The representation of such expression constructs with linear, covalently closed topology is shown in the European patent EP 0 941 318 B1. These dumbbell-shaped constructs have only the sequence information necessary for the expression of the target gene and by this avoid the disadvantages of plasmids with bacterial sequences. One possible disadvantage of the dumbbell-shaped expression constructs is that they induce relatively low expression of therapeutic proteins in comparison with plasmids of circular closed DNA double strands.

For the induction of an immune response, as is wanted during prophylactic or therapeutic vaccination, these linear vectors are clearly better than conventional plasmids, because they cause a significantly better immune response, both in quality and quantity (Lutz et al., 2000, J Virol: 74(22):10447-57).

Ligand arranged gene transfer is generally known to increase transfection efficiency. Transfer mediating ligands are bound to the vectors as, for instance, the nucleus or nuclear localization signal (NLS, amino acid sequence PKKKRKV) from SV-40 virus or the trans-activator protein TAT of HIV-1 virus, to enforce entry through the cell membrane and later into the cell core. This is a trial to mimic viral mechanisms of successful entry into cells and to equip non-viral vectors with efficient target finding systems.

It was possible to demonstrate a ten- to fifteen-fold increase of the antibody titer during an immunization experiment after coupling of the NLS-peptide to an expression cassette coding for HBsAg (Hepatitis small surface Antigen) in comparison to an uncoupled expression cassette (Schirmbeck et al., J. Mol. Med. 2001 June; 79 (5-6):343-50). A disadvantage is that the expression rate is clearly lower compared to vectors of the state of art.

In summary, it should be pointed out that despite intensive research, virtually no DNA vectors have been developed for use in gene therapy or genetic vaccination of humans or animals that have high transfection efficiency and, at the same time, are usable without security risks.

### OBJECT OR OBJECTS

Coming from this state of art, it is an objective of the present application to disclose a method for producing non-viral expression constructs with a high transfer and expression efficiency, as well as the corresponding expression construct itself and the options for its application. Thereby, the disadvantages of known viral expression systems, used within the scope of gene therapy, should be avoided.

### SUMMARY

The present application solves this technical problem, according to at least one embodiment, by a method for the production of a circular, bacterial and viral DNA free annular (circular) DNA expression construct, capable of transfecting cells efficiently and directed. According to at least one embodiment, the method comprises a method for the production of a circular, annular closed expression construct from a DNA double strand, comprising the following steps:
a) cleavage of a double stranded DNA sequence by a primary digestion with restriction endonucleases from a plasmid, which is amplifiable in prokaryotic or eukaryotic cells,
b) where the recognition sites limit the sequences of an expression cassette comprising
   i. at least one promoter sequence,
   ii. at least one coding sequence, and
   iii. at least one poly-adenylation sequence, directly, without any in-between located bases, on both sites, and
c) subsequent intramolecular ligation of the produced restriction fragments, so that a covalently closed DNA double strand develops (annulated closing) from the ligation reaction, followed by
d) a secondary digestion of the restriction mixture with a restriction endonuclease cutting a recognition sequence not present on the expression construct to be produced, but at least once present on the rest of the biological amplifiable plasmid, and
e) concurrent or following degradation of the unclosed rest of the biological amplifiable plasmid with an exonuclease specific for 3′- and 5′-DNA ends and
f) purification of the annular closed expression cassette from a DNA double strand.

As described in this application, a circular expression construct means a DNA vector comprising only a circular DNA double strand. The DNA double strand comprises at least one expression cassette, whereas an expression cassette comprises at least one promoter, at least one coding sequence and, if necessary, at least one poly-adenylation sequence. Appropriately, the vector has no free ends for protection against exonuclease degradation, but is annular closed.

The present application also solves this technical problem, according to at least one other embodiment, by a method for the production of a circular, annular closed expression construct from a DNA double strand, comprising the following steps:
a) cleavage of a double stranded DNA sequence by a primary digestion with restriction endonucleases from a plasmid, which is amplifiable in prokaryotic or eukaryotic cells,
b) where the recognition sites limit the sequences of an expression cassette comprising
   i. at least one promoter sequence,
   ii. at least one coding sequence, and
   iii. at least one poly-adenylation sequence, directly, without any in-between located bases, on both sites, and
c) subsequent intramolecular ligation of the produced restriction fragments in the presence of at least one oligodeoxynucleotide to that at least one ligand is bound covalently via chemical modifications, so that a covalently closed DNA double strand develops (annulated closing) under incorporation of the oligodeoxynucleotide, followed by
d) a secondary digestion of the restriction mixture with a restriction endonuclease cutting a recognition sequence not present on the expression construct to be produced, but at least once present on the rest of the biological amplifiable plasmid, and
e) concurrent or following degradation of the unclosed rest of the biological amplifiable plasmid with an exonuclease specific for 3′- and 5′-DNA ends and
f) purification of the annular closed expression cassette from a DNA double strand.

In a further embodiment according to the preceding embodiment, the vector is covalently closed by using an oligodeoxynucleotide that closes covalently the cohesive fragment ends of the digested expression cassette.

This oligodeoxynucleotide can have one or more modified bases allowing the coupling of one or more ligands.

Depending on the sequence of the used oligodeoxynucleotide, discrete structure motifs can form, allowing a freer and by this better access to the ligands. The designation “ligand” encloses in this context also peptides, proteins and/or other organic molecules like sugars or steroid molecules covalently coupled to the vector that lead to a directed and effective transfection of the cells.

Transfection means the introduction of nucleic acid sequences by biological, chemical or physical methods into the cell, causing an attenuated or temporary expression of proteins coded by these sequences or catalytically effective RNA transcripts within the transfected cell. It is also possible to introduce so-called “anti-sense” constructs, which inhibit protein expression by hybridization with complementary messenger RNAs.

By a method according to at least one embodiment, circular and ligand modified circular gene expression constructs are produced out of a continuous DNA double strand. Because the expression cassettes of these expression constructs according to at least one embodiment are absolutely limited to the control mechanisms necessary for the expression of therapeutic genes, only exactly defined nucleotides are present within the sequence of the vector neither with bacterial nor with viral origin. By this the vector size is reduced about 2 kilo bases and coincides with a reduction of CpG-sequence content of around about 90%. Expression constructs produced according to the method of at least one embodiment are not amplifiable, which means it is not possible to reproduce them in prokaryotic or in eucaryotic cells.

Circular expression constructs according to at least one embodiment are isolated by cleavage with type IIS restriction enzymes, preferably Eco31l, from a suitable plasmid containing the gene sequences to be expressed. The resulting cohesive fragment that contains the expression cassette will be closed by ligation to an annulus. In one embodiment one or more ligands are coupled to an oligodeoxynucleotide (ODN) that connects the cohesive fragment ends of the cleaved expression cassette. For the production of this peptide coupled vectors one or more transfer mediating ligands are coupled by covalent binding to the ODN before. The ODN is chemically modifiable by one or more alkalized carbonic acid, amine, thiol or aldehyde functions.

The plasmid backbone that is cleaved in a second restriction digestion by a restriction endonuclease, for which no recognition site is present in the expression cassette, will be enzymatically degraded by the exonuclease function of T7-polymerase, and the resting circular double stranded closed DNA expression construct according to at least one embodiment is purified chromatographically. Additionally, the expression construct can be purified by isopropanol precipitation. By the method according to at least one embodiment all sequence elements necessary for plasmid production, like bacterial or viral sequences, are eliminated.

By this reduction, except for sequence information necessary for expression, it is possible to produce a vector according to at least one embodiment in a novel smallness. So the production of vectors like these is possible in a range of 200-10000 bp, preferably 1000-2500 bp. In comparison, the average size of a common plasmid without coding sequences is 2000-3000 bp, and it is mostly impossible to fall below this size.

The covalent coupling of ligands to the ODN is done by a linker molecule and resembles by this a defined chemical binding. It is an advantage that the ligands are ligated to defined positions at the DNA vector. A possible loss of function of the promotor or of functional genes by binding of ligands within a functional sensitive region of the sequence is thereby excluded. Likewise it is advantageously possible to ligate multiple ligands independent from each other at defined places to the expression vector.

The present method according to at least one embodiment can also be present in form of a kit for a simple commercial application for the production of expression constructs according to at least one embodiment. Such a kit comprises:
(a) A plasmid, which
   contains the necessary recognition sequences for the restriction enzymes, and
   is suitable for amplification of the expression cassette,
(b) a first enzyme mix comprising restriction enzymes and ligase,
(c) a second enzyme mix comprising restriction enzymes and polymerase,
(d) basically known means for purification of the expression construct,
(e) commonly used reaction media, like buffer, ATP, DTT, water etc., and
(f) a control vector for function control of the elements of the kit.

Prerequisite for such a kit is the presence of a coding (arbitrary) sequence that is cloned by conventional methods into the plasmid (a).

Further advantages of additional embodiments are described herein. The surprising effect of circular, ligand modified double stranded DNA vectors according to at least one embodiment will be described by figures and examples.

The above-discussed embodiments of the present invention will be described further hereinbelow. When the word “invention” or “embodiment of the invention” is used in this specification, the word “invention” or “embodiment of the invention” includes “inventions” or “embodiments of the invention”, that is the plural of “invention” or “embodiment of the invention”. By stating “invention” or “embodiment of the invention”, the Applicants do not in any way admit that the present application does not include more than one patentably and non-obviously distinct invention, and maintains that this application may include more than one patentably and non-obviously distinct invention. The Applicants hereby assert that the disclosure of this application may include more than one invention, and, in the event that there is more than one invention, that these inventions may be patentable and non-obvious one with respect to the other.

### BRIEF DESCRIPTION OF THE DRAWINGS

FIG. 1 shows a schematic representation of the production procedure for circular DNA vectors according to at least one embodiment. In FIG. 1:
(a) plasmid;
(b) Eco 31l;
(c) ligand modified oligonucleotide linker;
(d) expression cassette;
(e) bacterial residual DNA;
(f) Eco 31l;
(g) mixture of bacterial residual DNA [symbol] and product (i);
(h) T7 DNA-polymerase; and
(i) product.

FIG. 2 shows a functional assembly of peptide coupled DNA vectors according to at least one embodiment. In FIG. 2:
(a) sequence of the gene to be expressed;
(b) promotor region;
(c) ligand modified oligonucleotide linker; and
(d) poly(A)-signal.

FIG. 3 is a graph showing an in vitro comparison of expression of a vector according to at least one embodiment, conventional plasmid and a linear vector with firefly-luciferase expression after transfection of K562 cells as measured by rlu (relative light units). In FIG. 3:

| | |
|---|---|
| plasmid | plasmid pMOK coding for luciferase; |
| lin vector | linear vector, coding for luciferase; and |
| circ vector2NLS | circular vector according to the invention |
| | couples to two NLS peptides. |

FIG. 4 is a graph showing an in vitro comparison of expression of a vector according to at least one embodiment, conventional plasmid and a linear vector with firefly-luciferase expression after transfection of HeLa cells as measured by rlu (relative light units). In FIG. 4:

| | |
|---|---|
| plasmid | plasmid pMOK, coding for luciferase; |
| lin vector | linear vector, coding for luciferase; and |
| circ vector | circular vector according to the invention |
| | without coupled peptides. |

FIG. 5 is a graph showing in vivo expression of Lac-Z coding circular and linear vectors. In FIG. 5:

| | |
|---|---|
| lin vector | linear vector, coding for Lac-Z; |
| lin vector-NLS | linear, NLS peptid coupled vector, coding |
| | for Lac-Z; and |
| circ vector-NLS | circular, NLS peptide coupled vector, |
| | according to at least one embodiment, |
| | coding for Lac-Z. |

FIG. 6 is a graph showing interferon-gamma secreting stimulated splenocytes in mice. In FIG. 6:

| | |
|---|---|
| lin vector-NLS | linear, NLS peptide coupled vector, coding |
| | for HBsAg; and |
| circ vector-NLS | circular, NLS peptide coupled vector |
| | according to at least one embodiment, |
| | coding for HBsAg. |

### DESCRIPTION OF EMBODIMENT OR EMBODIMENTS

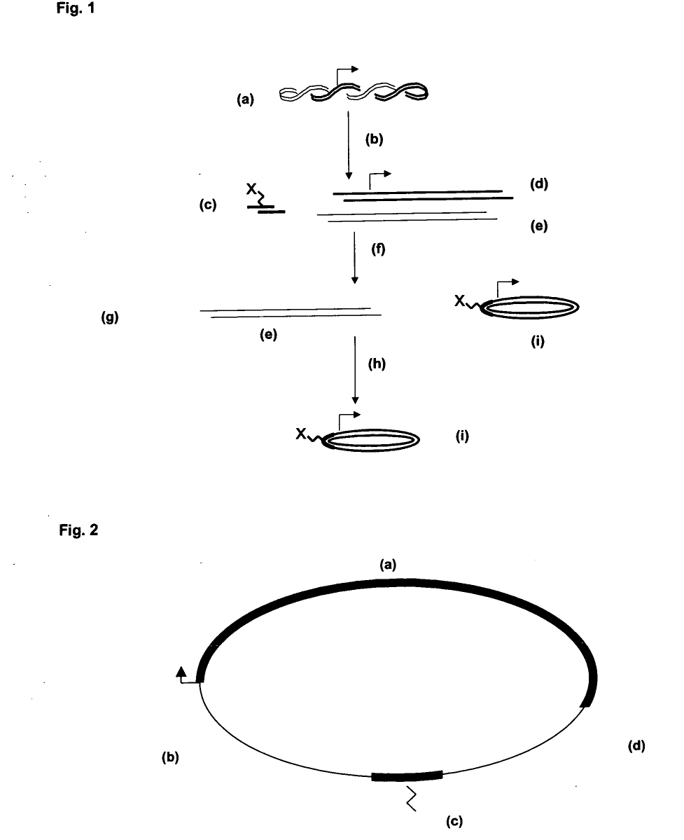

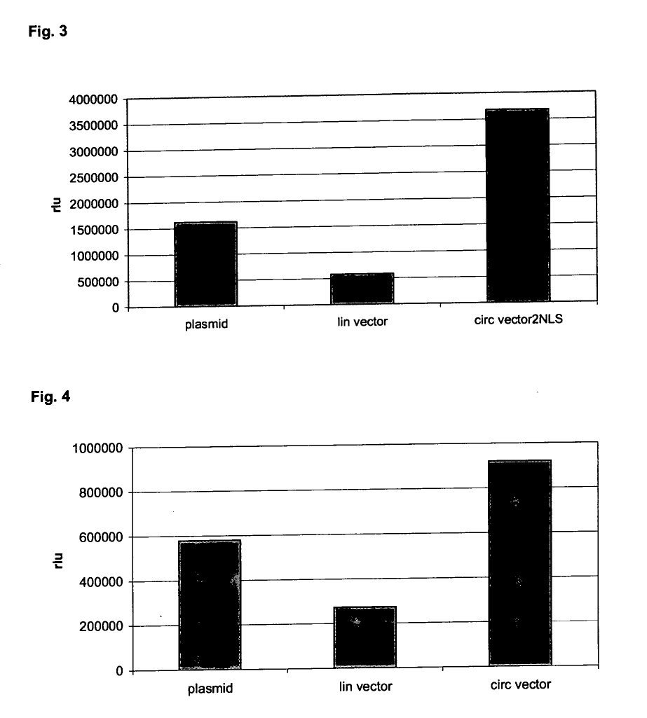

The expression verification with the reporter gene luciferase was done in vitro in two human cell lines. For comparison of the expression strength a plasmid coding for luciferase, a conventional vector and a vector according to the invention with and without peptide coupling were used. Surprisingly with the construct according to at least one embodiment with peptide modification NLS (nuclear or nucleus localization sequence, amino acid sequence PKKKRKV from SV-40 virus) an expression rate more than twice as high as with conventional plasmid was reached (see FIG. 3). Moreover a circular vector according to at least one embodiment without peptide modification shows a clear expression advantage over vectors of the state of art in FIG. 4.

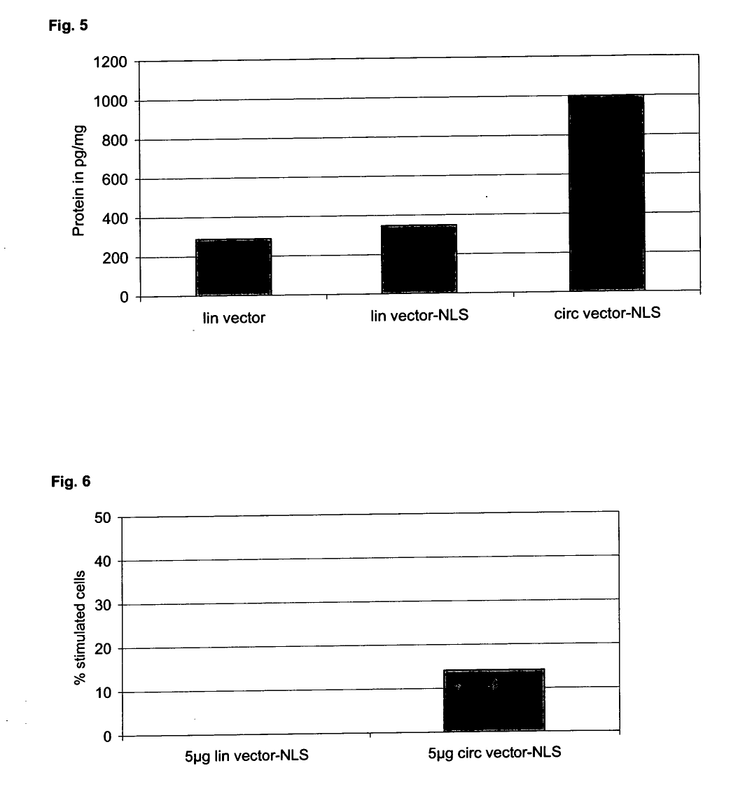

For in vivo studies, vectors according to at least one embodiment or commonly known ones, both coding for Lac-Z gene, were applied to mice. On the basis of quantitative β-galactosidase expression a comparison of vector induced expression of β-galactosidase expression was done. The vector according to at least one embodiment (as “circ vector-NLS” designated) is here also clearly advantageous. Like the in vitro results shown in FIG. 3, the in vivo expression rate is also increased about more than 50% (see FIG. 5).

A further in vivo experiment was made to investigate the immunologic courses after vaccination with linear and vectors according to at least one embodiment. For this mice were vaccinated with vectors coding for HBsAg and the resulting cytokine profile was compared on the basis of interferon-γ distribution. Interferon-γ plays a crucial role in the immune response and the anti-viral defense. Vectors according to at least one embodiment as shown in FIG. 6 are able to induce IFN-γ secreting splenocytes, as linear vectors are not able to cause IFN-γ secretion with the low amount of 5 μg vector used. For vaccination or immune therapy of different diseases, the induction of a Th1-typical immune response seems to be advantageous, especially for intracellular parasites like Leishmania and Malaria as well as viral caused diseases like HIV.

Besides the advantages of a significant increase of gene expression and the induction of a stronger cellular mediated immune response, these new kinds of vectors contain no marker genes or coding sequences with viral or bacterial origin, but only sequences directly necessary for expression of the therapeutic genes and guarantee by this maximal possible security for patients. On one hand unwanted immunologic or inflammatory processes are avoided as caused by bacterial or viral DNA, and on the other hand the correlating decrease of vector size seems to lead in an advantageous manner to an increased transfer rate into the cell core.

### EXAMPLE 1

### Production of Circular Vectors

The plasmid pMOK-Luc was completely digested with the restriction enzyme Eco3 μl for 2 h at 37° C. The restriction digestion created two DNA fragments. One comprised the canamycin resistance gene as well as other sequences necessary for plasmid propagation, and the other fragment consisted of the sequences that should form the vector according to at least one embodiment, namely CMV promotor, the gene sequence to be expressed and the polyadenylation sequence from SV-40. By the enzyme T4-DNA-ligase (in ligase buffer: 400 mM Tris-HCL, 100 mM MgCL₂, 5 mM ATP) the complementary ends produced by Eco31l were ligated over night at 4° C. to each other. The resulting mixture of nucleic acids was treated with the enzyme Eco147l. For degradation of resting DNA with vector origin the enzyme T7 DNA polymerase was added to the mixture. The remaining circular expression cassette was purified by anion exchange chromatography and was precipitated with isopropanol.

FIG. 1 shows a schematic representation of the production process of circular vectors according to at least one embodiment: By digestion of the plasmid (a) with Eco31l (b) the fragments of bacterial rest DNA (e) and the expression cassette are produced. Via the enzyme T4 DNA ligase (f) and in the presence of Eco31l (f) the ligand modified oligonucleotide linker (c) is ligated with (d). This mixture of bacterial rest DNA (g) and product (i) is treated in the last step (h) with T7 DNA polymerase and leads to the product (i).

### EXAMPLE 2a

### Ligand Coupling

Circular expression cassettes with coupled peptides were constructed as follows: The NLS peptide PKKKRKV (poline-lysine-lysine-lysine-arginine-lysine-valina) was coupled in two steps whether to one or both oligonucleotides. First the modified oligonucleotide (5′-PH-d(GGGAACCTTCAGTxAGCAATGG respectively 5′-PH-d AGGGCCATTGCTxACTGAAGG, where xT represents a amino-modified thymine-base with C2 amino-linker) (0.1 mM) was activated with sulfo-KMUS (5 mM) in coupling buffer (50 mM NaPO₄ and 75 mM NaCl, 0.5×, pH 7.6) at 37° C. for 2 h. The reaction was stopped with 50 mM Tris(hydroxymethyl)aminomethane (pH 7.5) and the activated ODN was received after ethanol precipitation (300 mM NaOAc pH 5.2, 5.5 mM MgCl₂, 100% ethanol), centrifugation and a single washing step with 70% ethanol. The ODN (0.1 mM) received by this was solved in coupling buffer (50 mM NaPO₄ und 75 mM NaCl, 0.5×, pH7.0) and reacted with the peptide (0.2 mM) for one hour at 37° C. The reaction was checked by a denaturing polyacrylamide gel (20%) and ethidium bromide staining. The resulting NLS coupled peptide was purified by HPLC and used for synthesis of the circular expression constructs.

### EXAMPLE 2b

### Production of Circular Vectors with Peptide Coupling

The plasmid pMOK-Luc was completely digested with the restriction enzyme Eco31l for 2 h at 37° C. The restriction digestion created two DNA fragments. By the enzyme T4 DNA ligase the previously at 90° C. or 3 min hybridized, complementary, 5′-phosphorylated oligodeoxynucleotides (TIBMolBiol, Berlin) 5′-PH-GGGAACCTTCAGTxAGCAATGG-3′ and 5′ PH-AGGGCCATTGCTxACTGAAGG-3′ (xT represents an amino-modified thymine-base with C2 linker, to which by choice the signal peptide NLS was covalently coupled) was ligated in presence of the restriction enzyme Eco31l over night at 4° C. to the vector forming fragment (compare example 1). The resulting mixture of nucleic acids was treated with the enzyme T7 DNA polymerase. The product was purified by anion exchange chromatography and precipitated with isopropanol.

### EXAMPLE 3

### Transfection of Cells, Expression Detection

Cells of the cell line K562 were transfected with the plasmid pMOK-Luc, a linear vector and a vector according to at least one embodiment with coupled ligand by electroporation. The experiment was done in triplicate; whereas after previous determination of concentration 100 ng DNA each was used. For each preparation 2.5×10⁶ cells/250 μl were used, transfected at 300V and 1050 μF. After incubation for 24 hours at 37° C. the expression detection was done by determination of luciferase activity. The results are shown in FIG. 3.

### EXAMPLE 4

### Transfection with Lipofektin

The day before transfection in 24-well plates 40,000 cells of the human cell line HeLa were seeded (20,000 cells/cm²). The cells were transfected with Lipofektin using the following DNA: plasmid pMOK, linear vector, circular vector without coupled peptide; each coding for the reporter gene luciferase. The experiment was done 4- to 8-fold, whereas after previous determination of concentration 800 ng DNA each was used. The DNA was incubated with the transfection reagent Lipofektin in DMEM (Dulbecco's Modified Eagle Medium) 45 at 20° C. Afterwards the cells were transfected by addition of the DNA-Lipofektin mixture to the cells. The duration of incubation was 4 h at 37° C. After exchange of the medium the cells were further cultivated for 21 h at 37° C., followed by determination of expression via control of luciferase activity in a luminumeter. The results are displayed in FIG. 4.

### EXAMPLE 5

### Intratumoral Injection of Lac-Z Coding Vectors

The groups of six mice each were intratumorally injected by a jet-injection-method with linear vector and circular vector with or without coupled peptide coding for the Lac-Z gene. The animals received five injections. The DNA concentration was each 1 μg/μl. 48 h after the last injection the animals were killed, the tumor was removed, and stored in liquid nitrogen for further clinical examinations. The preparation of tumor cells was done by homogenization in 800 μl lysis buffer (TE-Buffer, pH 8, containing aprotinine 10 mg/ml and PMSF 10 μg/ml). After centrifugation (14000 rpm, 4° C., 10 min), the lysate was received and the protein determination was performed by Coomassie staining (Pierce, Rockford, USA). The absorption was determined at 595 nm. The results are shown in FIG. 5.

### EXAMPLE 6

### Interferon-γ Secretion after Immunization with HBsAg

Six to eight week old BALB/c mice were intradermally immunized with vector coding for HBsAg (solved in 50 μl 100 mM Na₂PO₄). After four weeks, from two mice of each group the spleen was received and the splenocytes were isolated. The splenocytes were incubated with concanavalin A (ConA), mitomycin C treated antigen presenting cells (APC, as negative control) and with APCs that were in contact with HBsAg peptide (positive control) over night at 37° C. with 5% CO₂. The 96-well plates were coated previously with 8 μg/ml anti-mouse IFN-γ antibody (Pharmingen). After incubation the cells were removed and 100 μl biotinylated anti-mouse IFN-γ antibody (Pharmingen) with a concentration of 2 μg/ml was added to the 96-well plates. After incubation over night at 4° C., the plates were washed and incubated for 1 hour at room temperature after addition of 100 μl of a 1:800 dilution of avidin-peroxidase. Development was started by addition of 100 μl DAB substrate (Sigma). After 20 min the reaction was stopped and the precipitates were counted using a stereo-microscope. The results of the negative control were subtracted from the results of the positive control and by this the number of antigen specific precipitates determined. These values were set in relation to the results of the ConA incubated splenocytes. The results are displayed in FIG. 6.

One feature or aspect of an embodiment is believed at the time of the filing of this patent application to possibly reside broadly in a method for the production of a circular, annular closed expression construct from a DNA double strand, comprising the following steps: cleavage of a double stranded DNA sequence by a primary digestion with restriction endonucleases from a plasmid, which is amplifiable in prokaryotic or eukaryotic cells; where the recognition sites limit the sequences of an expression cassette comprising: at least one promoter sequence, at least one coding sequence, and at least one poly-adenylation sequence, directly, without any in-between located bases, on both sites; and subsequent intramolecular ligation of the produced restriction fragments, so that a covalently closed DNA double strand develops (annulated closing) from the ligation reaction, followed by a secondary digestion of the restriction mixture with a restriction endonuclease cutting a recognition sequence not present on the expression construct to be produced, but at least once present on the rest of the biological amplifiable plasmid, and concurrent or following degradation of the unclosed rest of the biological amplifiable plasmid with an exonuclease specific for 3′- and 5′-DNA ends and purification of the annular closed expression cassette from a DNA double strand.

Another feature or aspect of an embodiment is believed at the time of the filing of this patent application to possibly reside broadly in a method for the production of a circular, annular closed expression construct from a DNA double strand, comprising the following steps: cleavage of a double stranded DNA sequence by a primary digestion with restriction endonucleases from a plasmid, which is amplifiable in prokaryotic or eukaryotic cells, where the recognition sites limit the sequences of an expression cassette comprising: at least one promoter sequence; at least one coding sequence; and at least one poly-adenylation sequence, directly, without any in-between located bases, on both sites, and subsequent intramolecular ligation of the produced restriction fragments in the presence of at least one oligodeoxynucleotide to that at least one ligand is bound covalently via chemical modifications, so that a covalently closed DNA double strand develops (annulated closing) under incorporation of the oligodeoxynucleotide, followed by a secondary digestion of the restriction mixture with a restriction endonuclease cutting a recognition sequence not present on the expression construct to be produced, but at least once present on the rest of the biological amplifiable plasmid, and concurrent or following degradation of the unclosed rest of the biological amplifiable plasmid with an exonuclease specific for 3′- and 5′-DNA ends and purification of the annular closed expression cassette from a DNA double strand.

Yet another feature or aspect of an embodiment is believed at the time of the filing of this patent application to possibly reside broadly in a method where the oligodeoxynucleotide is chemical modified by one or more carbonic acid, amine, thiol, or aldehyde function.

Still another feature or aspect of an embodiment is believed at the time of the filing of this patent application to possibly reside broadly in a method where the primary restriction digestion is done by one or more type IIS restriction endonucleases, preferably Eco31l.

A further feature or aspect of an embodiment is believed at the time of the filing of this patent application to possibly reside broadly in a method where the secondary restriction digestion is done preferably with the enzyme Eco147l.

One feature or aspect of an embodiment is believed at the time of the filing of this patent application to possibly reside broadly in an expression construct for the transport of genetic information, comprising double stranded DNA, where the expression construct is circular, annular closed and has no bacterial and/or viral sequences, further the expression construct is not amplifiable in prokaryotic or eukaryotic cells, as well as the expression construct consists at least of an expression cassette of double stranded DNA, and where a expression cassette comprises: at least one promoter sequence, at least one coding sequence, and at least one polyadenylation sequence, and the expression construct spans 200 to 10,000 bp.

Another feature or aspect of an embodiment is believed at the time of the filing of this patent application to possibly reside broadly in an expression construct where the expression construct spans at least 1000 to 2500 bp.

Yet another feature or aspect of an embodiment is believed at the time of the filing of this patent application to possibly reside broadly in an expression construct where the expression construct contains at least one oligodeoxynucleotide.

Still another feature or aspect of an embodiment is believed at the time of the filing of this patent application to possibly reside broadly in an expression construct where the oligodeoxynucleotide has at least one amino-modified thymine-base.

A further feature or aspect of an embodiment is believed at the time of the filing of this patent application to possibly reside broadly in an expression construct where the oligodeoxynucleotide is chemically modifiable by one or more carbonic acid, amine, thiol or aldehyde functions.

Another feature or aspect of an embodiment is believed at the time of the filing of this patent application to possibly reside broadly in an expression construct wheat at least one ligand is covalently bound to the oligodeoxynucleotide.

Yet another feature or aspect of an embodiment is believed at the time of the filing of this patent application to possibly reside broadly in an expression construct where the ligand is an oligo-peptide.

Still another feature or aspect of an embodiment is believed at the time of the filing of this patent application to possibly reside broadly in an expression construct where the oligo-peptide consists of 3 to 30 amino acids, where at least half are the basic amino acids arginine and/or lysine.

A further feature or aspect of an embodiment is believed at the time of the filing of this patent application to possibly reside broadly in an expression construct where the oligo-peptide has a nucleus localization sequence with amino acid sequence PKKKRKV.

One feature or aspect of an embodiment is believed at the time of the filing of this patent application to possibly reside broadly in use of an expression construct for transport of genetic information for the gene therapeutic application in humans or animals.

One feature or aspect of an embodiment is believed at the time of the filing of this patent application to possibly reside broadly in use of an expression construct as vaccine.

One feature or aspect of an embodiment is believed at the time of the filing of this patent application to possibly reside broadly in use of an expression construct as part of a kit.

The components disclosed in the various publications, disclosed or incorporated by reference herein, may possibly be used in possible embodiments of the present invention, as well as equivalents thereof.

At least one embodiment of the invention relates to a method for producing a circular minimalist expression construct closed in an annular manner, from a double-strand DNA, to the expression construct produced according to said method, and to the use of the same. The inventive expression construct can be covalently modified and used for the effective and targeted transfection of cells in gene therapy.

The purpose of the statements about the technical field is generally to enable the Patent and Trademark Office and the public to determine quickly, from a cursory inspection, the nature of this patent application. The description of the technical field is believed, at the time of the filing of this patent application, to adequately describe the technical field of this patent application. However, the description of the technical field may not be completely applicable to the claims as originally filed in this patent application, as amended during prosecution of this patent application, and as ultimately allowed in any patent issuing from this patent application. Therefore, any statements made relating to the technical field are not intended to limit the claims in any manner and should not be interpreted as limiting the claims in any manner.

The appended drawings in their entirety, including all dimensions, proportions and/or shapes in at least one embodiment of the invention, are accurate and are hereby included by reference into this specification.

The background information is believed, at the time of the filing of this patent application, to adequately provide background information for this patent application. However, the background information may not be completely applicable to the claims as originally filed in this patent application, as amended during prosecution of this patent application, and as ultimately allowed in any patent issuing from this patent application. Therefore, any statements made relating to the background information are not intended to limit the claims in any manner and should not be interpreted as limiting the claims in any manner.

All, or substantially all, of the components and methods of the various embodiments may be used with at least one embodiment or all of the embodiments, if more than one embodiment is described herein.

The purpose of the statements about the object or objects is generally to enable the Patent and Trademark Office and the public to determine quickly, from a cursory inspection, the nature of this patent application. The description of the object or objects is believed, at the time of the filing of this patent application, to adequately describe the object or objects of this patent application. However, the description of the object or objects may not be completely applicable to the claims as originally filed in this patent application, as amended during prosecution of this patent application, and as ultimately allowed in any patent issuing from this patent application. Therefore, any statements made relating to the object or objects are not intended to limit the claims in any manner and should not be interpreted as limiting the claims in any manner.

All of the patents, patent applications and publications recited herein, and in the Declaration attached hereto, are hereby incorporated by reference as if set forth in their entirety herein.

The summary is believed, at the time of the filing of this patent application, to adequately summarize this patent application. However, portions or all of the information contained in the summary may not be completely applicable to the claims as originally filed in this patent application, as amended during prosecution of this patent application, and as ultimately allowed in any patent issuing from this patent application. Therefore, any statements made relating to the summary are not intended to limit the claims in any manner and should not be interpreted as limiting the claims in any manner.

It will be understood that the examples of patents, published patent applications, and other documents which are included in this application and which are referred to in paragraphs which state “Some examples of . . . which may possibly be used in at least one possible embodiment of the present application . . . ” may possibly not be used or useable in any one or more embodiments of the application.

The sentence immediately above relates to patents, published patent applications and other documents either incorporated by reference or not incorporated by reference.

All of the patents, patent applications or patent publications, which were cited in the International Search Report mailed Oct. 31, 2003, and/or cited elsewhere are hereby incorporated by reference as if set forth in their entirety herein as follows: WO 96 05297 A (SEEBER, STEFAN; RUEGER, RUEDIGER (DE); BOEHRINGER MANNHEIM GMBH (DE)) 22 Feb. 1996; U.S. Pat. No. 6,143,530 A (WILS, PIERRE ET AL) 7 Nov. 2000; WO 98 21322 A (JUNGHANS, CLAAS; SOFT GENE GMBH (DE); WITTIG, BURGHARDT (DE)) 22 May 1998; EP 0 967 274 A (MOLOGEN GMBH) 29 Dec. 1999; LOPEZ-FUERTES L ET AL: “DNA vaccination with linear minimalistic (MIDGE) vectors confers protection against Leishmania major infection in mice” VACCINE, BUTTERWORTH SCIENTIFIC. GUILDFORD, GB, vol. 21, no. 3-4, 13 Dec. 2002; SCHIRMBECK R ET AL: “Priming of immune responses to hepatitis B surface antigen with minimal DNA expression constructs modified with a nuclear localization signal peptide” JOURNAL OF MOLECULAR MEDICINE, SPRINGER VERLAG, DE, vol. 79, no. 5-6, June 2001; JOHANSSON P ET AL: “PCR-generated linear DNA fragments utilized as a hantavirus DNA vaccine” VACCINE, BUTTERWORTH SCIENTIFIC. GUILDFORD, GB, vol. 20, no. 27-28, 10 Sep. 2002; GURUNATHAN S ET AL: “DNA vaccines: immunology, application, and optimization*.” ANNUAL REVIEW OF IMMUNOLOGY. UNITED STATES 2000, vol. 18, 2000, pages 927-974; and WO 94 09127 A (US HEALTH) 28 Apr. 1994 (1994-04-28) abstract.

The corresponding international patent publication application, namely, International Application No. PCT/DE2003/001970, filed Jun. 10, 2003, having WIPO Publication No. WO 2004/111247 and publication date of Dec. 23, 2004, and having inventors Matthias SCHROFF and Colin SMITH, is hereby incorporated by reference as if set forth in their entirety herein for the purpose of correcting and explaining any possible misinterpretations of the English translation thereof. In addition, the published equivalents of the above corresponding foreign and international patent publication applications, and other equivalents or corresponding applications, if any, in corresponding cases in the Federal Republic of Germany and elsewhere, and the references and documents cited in any of the documents cited herein, such as the patents, patent applications and publications, are hereby incorporated by reference as if set forth in their entirety herein.

All of the references and documents, cited in any of the documents cited herein, are hereby incorporated by reference as if set forth in their entirety herein. All of the documents cited herein, referred to in the immediately preceding sentence, include all of the patents, patent applications and publications cited anywhere in the present application.

The description of the embodiment or embodiments is believed, at the time of the filing of this patent application, to adequately describe the embodiment or embodiments of this patent application. However, portions of the description of the embodiment or embodiments may not be completely applicable to the claims as originally filed in this patent application, as amended during prosecution of this patent application, and as ultimately allowed in any patent issuing from this patent application. Therefore, any statements made relating to the embodiment or embodiments are not intended to limit the claims in any manner and should not be interpreted as limiting the claims in any manner.

The details in the patents, patent applications and publications may be considered to be incorporable, at applicant's option, into the claims during prosecution as further limitations in the claims to patentably distinguish any amended claims from any applied prior art.

The purpose of the title of this patent application is generally to enable the Patent and Trademark Office and the public to determine quickly, from a cursory inspection, the nature of this patent application. The title is believed, at the time of the filing of this patent application, to adequately reflect the general nature of this patent application. However, the title may not be completely applicable to the technical field, the object or objects, the summary, the description of the embodiment or embodiments, and the claims as originally filed in this patent application, as amended during prosecution of this patent application, and as ultimately allowed in any patent issuing from this patent application. Therefore, the title is not intended to limit the claims in any manner and should not be interpreted as limiting the claims in any manner.

The following U.S. Patents and Patent Applications are hereby incorporated by reference as if set forth in their entirety herein: U.S. Pat. No. 6,451,563, issued on Sep. 17, 2002; U.S. Pat. No. 6,451,593, issued on Sep. 17, 2002; U.S. Pat. No. 6,849,725, issued on Feb. 1, 2005; U.S. patent application Ser. No. 10/528,748, filed on Mar. 22, 2005; U.S. patent application Ser. No. 10/816,465, filed on Apr. 1, 2004; and U.S. patent application Ser. No. 10/816,591, filed on Apr. 1, 2004.

The abstract of the disclosure is submitted herewith as required by 37 C.F.R. §1.72(b). As stated in 37 C.F.R. §1.72(b):
A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims.
   Therefore, any statements made relating to the abstract are not intended to limit the claims in any manner and should not be interpreted as limiting the claims in any manner.

The embodiments of the invention described herein above in the context of the preferred embodiments are not to be taken as limiting the embodiments of the invention to all of the provided details thereof, since modifications and variations thereof may be made without departing from the spirit and scope of the embodiments of the invention.

1. A method for the production of a circular, annular closed expression construct from a DNA double strand, comprising the following steps:
a) cleavage of a double stranded DNA sequence by a primary digestion with restriction endonucleases from a plasmid, which is amplifiable in prokaryotic or eukaryotic cells,
b) where the recognition sites limit the sequences of an expression cassette comprising
i. at least one promoter sequence,
ii. at least one coding sequence, and
iii. at least one poly-adenylation sequence,
directly, without any in-between located bases, on both sites, and
c) subsequent intramolecular ligation of the produced restriction fragments, so that a covalently closed DNA double strand develops (annulated closing) from the ligation reaction, followed by
d) a secondary digestion of the restriction mixture with a restriction endonuclease cutting a recognition sequence not present on the expression construct to be produced, but at least once present on the rest of the biological amplifiable plasmid, and
e) concurrent or following degradation of the unclosed rest of the biological amplifiable plasmid with an exonuclease specific for 3′- and 5′-DNA ends and
f) purification of the annular closed expression cassette from a DNA double strand.

2. The method according to claim 1, where the primary restriction digestion is done by one or more type IIS restriction endonucleases, preferably Eco31l.

3. The method according to claim 1, where the secondary restriction digestion is done preferably with the enzyme Eco147l.

4. A method for the production of a circular, annular closed expression construct from a DNA double strand, comprising the following steps:
a) cleavage of a double stranded DNA sequence by a primary digestion with restriction endonucleases from a plasmid, which is amplifiable in prokaryotic or eukaryotic cells,
b) where the recognition sites limit the sequences of an expression cassette comprising
i. at least one promoter sequence,
ii. at least one coding sequence, and
iii. at least one poly-adenylation sequence,
directly, without any in-between located bases, on both sites, and
c) subsequent intramolecular ligation of the produced restriction fragments in the presence of at least one oligodeoxynucleotide to that at least one ligand is bound covalently via chemical modifications, so that a covalently closed DNA double strand develops (annulated closing) under incorporation of the oligodeoxynucleotide, followed by
d) a secondary digestion of the restriction mixture with a restriction endonuclease cutting a recognition sequence not present on the expression construct to be produced, but at least once present on the rest of the biological amplifiable plasmid, and
e) concurrent or following degradation of the unclosed rest of the biological amplifiable plasmid with an exonuclease specific for 3′- and 5′-DNA ends and
f) purification of the annular closed expression cassette from a DNA double strand.

5. The method according to claim 4, where the oligodeoxynucleotide is chemically modified by one or more carbonic acid, amine, thiol, or aldehyde function.

6. The method according to claim 4, where the primary restriction digestion is done by one or more type IIS restriction endonucleases, preferably Eco31l.

7. The method according to claim 4, where the secondary restriction digestion is done preferably with the enzyme Eco147l.

8. An expression construct for the transport of genetic information, comprising double stranded DNA, where
a) the expression construct is circular, annular closed and has no bacterial and/or viral sequences, further
b) the expression construct is not amplifiable in prokaryotic or eukaryotic cells, as well as
c) the expression construct comprises at least of an expression cassette of double stranded DNA, and where a expression cassette comprises
i. at least one promoter sequence,
ii. at least one coding sequence, and
iii. at least one polyadenylation sequence,
d) the expression construct spans 200 to 10,000 bp.

9. The expression construct according to claim 8, where the expression construct spans at least 1000 to 2500 bp.

10. The expression construct according to claim 9, where the expression construct contains at least one oligodeoxynucleotide.

11. The expression construct according to claim 10, where the oligodeoxynucleotide has at least one amino-modified thymine-base.

12. The expression construct according to claim 11, where the oligodeoxynucleotide is chemically modifiable by one or more carbonic acid, amine, thiol or aldehyde functions.

13. The expression construct produced according to claim 12, wherein at least one ligand is covalently bound to the oligodeoxynucleotide.

14. The expression construct according to claim 13, where the ligand is an oligo-peptide.

15. The expression construct according to claim 14, where the oligo-peptide comprises 3 to 30 amino acids, where at least half are the basic amino acids arginine and/or lysine.

16. The expression construct according to claim 14, where the oligo-peptide has a nuclear localization sequence with amino acid sequence PKKKRKV.

17. A use of the expression construct according to claim 16 for transport of genetic information for the gene therapeutic application in humans or animals.

18. A use of the expression construct according to claim 6 for transport of genetic information for the gene therapeutic application in humans or animals.

19. A use of the expression construct according to claim 6 as vaccine.

20. A use of the expression construct according to claim 6 as part of a kit.


SEQUENCE LISTING (3 sequences)

SEQ ID NO: 1 - protein, 7 aa, Simian virus 40
  MISC_FEATURE: NLS peptide
PKKKRKV

SEQ ID NO: 2 - DNA, 21 bp, Artificial
  ODN 1
gggaaccttcagtagcaatgg

SEQ ID NO: 3 - DNA, 20 bp, Artificial
  ODN 2
agggccattgctactgaagg